(19) United States
(12) Patent Application Publication
(10) Pub. No.: US 2017/0002297 A1
(43) Pub. Date: Jan. 5, 2017

(54) Glucamides In Syndet Soaps

(71) Applicant: CLARIANT INTERNATIONAL LTD., Muttenz (CH)

(72) Inventors: Peter KLUG, Grossostheim (DE); Gerd DAHMS, Duisburg (DE); Lisa Renne GANDOLFI, Charalotte, NC (US)

(73) Assignee: Clariant International Ltd., Muttenz (CH)

(21) Appl. No.: 15/100,165

(22) PCT Filed: Dec. 3, 2014
(86) PCT No.: PCT/EP2014/003215

§ 371 Date: May 27, 2016

Related U.S. Application Data
(60) Provisional application No. 61/911,003, filed on Dec. 3, 2013.

(51) Int. Cl. C11D 3/34; C11D 1/12; C11D 1/52; C11D 1/655; A61Q 19/00; A61K 8/41; A61K 8/36; A61K 8/46; A61Q 19/10; C11D 10/04; C11D 17/00

(52) U.S. Cl. CPC C11D 3/3472; C11D 10/04; C11D 1/126; C11D 1/525; C11D 1/655; C11D 17/006; C11D 17/0047; A61K 8/41; A61K 8/361; A61K 8/466; A61Q 19/10; A61Q 19/007

(57) ABSTRACT

Disclosed are compositions containing:—at least one N-alkyl-N-acyl glucamine as component A;—at least one fatty acid and/or soap as component B;—at least one acyl isethionate as component C;—sodium isethionate as component D;—water as component E; and—optionally, at least one other additive as component F. More than 20 weight percent, preferably more than 70 weight percent of the N-alkyl-N-acyl glucamines contain at least one C₁₂- and/or C₁₄- and/or C₁₆- and/or C₁₈-acyl group. Said compositions can be used as bars of soap.

The invention relates to a composition comprising N-alkyl-N-acylglucamines, fatty acids and/or soaps, acyl isethionates and sodium isethionate. In addition, the invention relates to the use of the composition as soap bar, to the use for the treatment or care of skin or hair, and to a process for producing a composition according to the invention.

Soap bars have always played a major role in body cleaning and care. Classic soaps in this connection are the so-called alkali soaps. Alkali soaps contain exclusively fatty acid salts and possibly also free fatty acids as impurity. The fatty acid salts are formed by reacting fatty acids with an alkali, e.g. potassium hydroxide solution or sodium hydroxide solution, in a saponification reaction.

As well as the classic alkali soaps, so-called combibars are nowadays also supplied. Combibars are soap bars which, as well as fatty acid salts, also have further synthetic surfactants (as a rule fatty alcohol ether sulfates or fatty acid isethionates).

The so-called syndet bars are becoming increasingly important. Syndet bars are soap bars which are free from fatty acid salts and have exclusively synthetic surfactants. They are attributed particularly good skin compatibility and a low allergy-triggering potential.

In general, high requirements are nowadays placed on soap bars. The soap bars should have not only a cleaning effect, but also care properties. A soap bar should thus have pleasing haptics and generate a particularly large amount of creamy lather upon use.

The object of the invention is thus to provide improved compositions which, particularly in the event of their use as soap bar, produce an increased amount of foam, have a greater hardness and lead to a reduction in roughness.

The use of N-alkyl-N-acylglucamines and isethionates in soap manufacture has already been known for a long time.

WO 98/05752 and WO 92/13059 disclose soap bars which have alkyl-N-methylglucamides.

WO 98/15606 describes fatty-acid-free syndet soaps which comprise fatty acid N-alkylglucamides and fatty acid isethionates.

WO 01/72946, DE 19645214 and WO 01/72947 describe soaps made of fatty acid N-alkylpolyhydroxyalkylamides and coconut fatty acid isethionate Na salt. The combination with coconut fatty acid isethionate NH₄ salt is disclosed in WO 95/07975.

WO 98/00492 and WO 98/06800 are directed to soap bars which comprise glucamides and acyl isethionates.

DE 19703745 also discloses soap bars. The compositions described here comprise fatty acid N-alkylglucamides and fatty acids.

It has now been found that compositions which comprise sodium isethionate as well as an N-alkyl-N-acylglucamine, a fatty acid and/or soap and an acyl isethionate are particularly suitable for use as soap bars. Such a suitability cannot be derived from any of the cited documents.

Accordingly, a composition is provided, comprising:
at least one N-alkyl-N-acylglucamine as component A,
at least one fatty acid and/or soap as component B,
at least one acyl isethionate as component C,
sodium isethionate as component D,
water as component E,
optionally at least one further additive as component F,

where more than 20% by weight, preferably more than 70% by weight, of the N-alkyl-N-acylglucamines contain at least one C₁₂- and/or C₁₄- and/or C₁₆- and/or C₁₈-acyl group.

In a preferred embodiment, in a composition according to the invention, the component A comprises N-alkyl-N-acylglucamines, where more than 20% by weight, preferably more than 70% by weight, of the N-alkyl-N-acylglucamines contain at least one C₁₂- and/or C₁₄-acyl group.

The composition according to the invention advantageously has increased lathering, increased hardness and reduced roughness.

According to the invention, preference is given to a composition comprising:
0.5-5.0% by weight of the composition of component A,
25.0-50.0% by weight of the composition of component B,
28.0-50.0% by weight of the composition of component C,
2.0-10.0% by weight of the composition of component D.

Particular preference is given to a composition comprising:
1.0-3.0% by weight of the composition of component A,
25.0-35.0% by weight of the composition of component B,
40.0-48.0% by weight of the composition of component C,
3.0-6.0% by weight of the composition of component D.

The N-alkyl-N-acylglucamines used according to the invention, in which glucamine is preferably an N-1-deoxysorbityl group, are particularly preferably N-alkyl-N-acylglucamines of the formula (I),

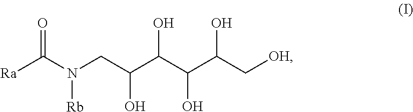

where, in the formula (I), RₐCO is a linear or branched, saturated or unsaturated C₈-C₂₂-acyl radical and R_{b} is a C₁-C₄ alkyl radical. Particularly preferably, R_{b} in formula (I) is a methyl radical (—CH₃) and RₐCO has the above meaning.

Preferred N-alkyl-N-acylglucamines are compounds of the formula (I) in which RₐCO is a C₁₂-C₁₈-acyl radical. Particular preference is given to N-alkyl-N-acylglucamines of the formula (I) in which RₐCO is a C₁₂-C₁₈-acyl radical and R_{b} is a methyl radical.

The fraction of N-alkyl-N-acylglucamines which contain a C₁₂- and/or C₁₄- and/or C₁₆- and/or C₁₈-acyl group, in particular a C₁₂- and/or C₁₄-acyl group, is particularly preferably at least 70% by weight and the fraction of N-alkyl-N-acylglucamines which contain an acyl group <C₁₂ is less than 3% by weight.

The fraction of N-alkyl-N-acylglucamines which contain a C₁₂- and/or C₁₄- and/or C₁₆- and/or C₁₈-acyl group, in particular a C₁₂- and/or C₁₄-acyl group, is particularly preferably at least 80% by weight. Preferably, at the same time the fraction of N-alkyl-N-acylglucamines which contain an acyl group <C₁₂ is less than 2% by weight.

In a further embodiment, the fraction of N-alkyl-N-acylglucamines which contain a C₁₂- and/or C₁₄- and/or C₁₆- and/or C₁₈-acyl group, in particular a C₁₂- and/or C₁₄-acyl group, is at least 90% by weight. Preferably, at the same time, the fraction of N-alkyl-N-acylglucamines which contain an acyl group <012 is less than 2% by weight.

Within the context of a further preferred embodiment, the component A consists of a mixture of N-alkyl-N-acylglucamines. Preferably, the mixture is a mixture which comprises at least one N-alkyl-N-C₈-C₂₂-acylglucamine, particularly preferably at least one N-methyl-N-C₈-C₂₂-acylglucamine.

Preferred components A are saturated N-alkyl-N-acylglucamines of the formula (I), where the acyl radical RₐCO is derived from myristic acid, lauric acid, palmitic acid, stearic acid, oleic acid, linoleic acid or linolenic acid.

Preference is also given to N-alkyl-N-acylglucamines of the formula (I) in which RₐCO is derived from coconut oil.

Coconut oil typically comprises triglycerides which contain saturated fatty acid radicals which are derived from caprylic acid, lauric acid, capric acid, oleic acid, palmitic acid, stearic acid and myristic acid.

Coconut oil preferably comprises in this connection

a) 40-55% by weight of lauric acid,

b) 10-20% by weight of myristic acid,

c) 8-12% by weight of palmitic acid,

d) 6-12% by weight of oleic acid and

h) 0-36% by weight of further fatty acids,

where the sum of the fatty acids bonded to the triglyceride is 100% by weight.

Particularly preferably, coconut oil comprises

a) 40-55% by weight of lauric acid,

b) 10-20% by weight of myristic acid,

c) 8-12% by weight of palmitic acid,

d) 6-12% by weight of acid,

e) 5-10% by weight of decanoic acid,

f) 4-10% by weight of octanoic acid,

g) 1-3% by weight of stearic acid and

h) 0-26% by weight of further fatty acids,

where the sum of the fatty acids bonded to the triglyceride is 100% by weight.

Besides the N-alkyl-N-acylglucamines of the formula (I) in which RₐCO is a C₁₂-C₁₄-acyl radical, the compositions can comprise small fractions of N-alkyl-N-acylglucamines derived from short-chain and/or long-chain fatty acids, in particular those which contain C₁-C₄-acyl, C₆-, C₈-, C₁₀-, C₁₆-, C₁₈- and/or C₂₀-acyl.

The weight ratio of N-alkyl-N-acylglucamine of the formula (I) where RₐCO is a C₁₂-alkyl radical to N-alkyl-N-acylglucamine of the formula (I) where RₐCO is a C₁₄-alkyl radical is particularly preferably 50:50 to 90:10, in particular 60:40 to 80:20.

In a further embodiment, the N-alkyl-N-acylglucamine of a composition according to the invention is a mixture of at least one N-alkyl-N-acylglucamine of the formula (I) where RₐCO is a C₁₆-acyl radical and at least one N-alkyl-N-acylglucamine of the formula (I), where RₐCO is a C₁₈-acyl radical.

Preferably, the fraction of component A in a composition according to the invention is 0.5-5.0% by weight, based on the composition, and particularly preferably 1.0-3.0% by weight, based on the composition.

The N-alkyl-N-acylglucamines used here can be prepared as described in EP 0 550 637 A1 by reacting the corresponding fatty acid esters or fatty acid ester mixtures with N-alkylglucamine in the presence of a solvent having hydroxyl groups or alkoxyl groups. Suitable solvents are, for example, C₁-C₄-monoalcohols, ethylene glycol, propylene glycol, glycerol, and alkoxylated alcohols. Preference is given to 1,2-propylene glycol. N-Alkylglucamine can be obtained, as likewise described in EP 0 550 637 A1, by reductive amination of glucose with alkylamine.

Suitable fatty acid esters which are reacted with the N-alkylglucamines to give N-alkyl-N-acylglucamines are generally the alkyl esters, specifically the corresponding methyl esters or ethyl esters, which are obtained by transesterification from natural fats and oils, for example the triglycerides.

Suitable raw materials for preparing the fatty acid alkyl esters are, for example, coconut oil or palm oil, with coconut oil being particularly preferred.

As further component, a composition according to the invention comprises at least one fatty acid and/or soap as component B.

The fatty acids of component B are preferably natural fatty acids, particularly preferably having 8 to 22 carbon atoms, such as, for example, octanoic acid, decanoic acid, lauric acid, myristic acid, palmitic acid, stearic acid, oleic acid, linoleic acid, linolenic acid, behenic acid, hydroxyl fatty acids, for example 12-hydroxystearic acid or 16-hydroxyhexadecanoic acid and mixtures thereof.

Particular preference is given to fatty acids having 12 to 18 carbon atoms.

The soaps of component B are salts of fatty acids, in particular alkali metal salts, preferably sodium or potassium salts. The fatty acids here have in particular 8 to 22 carbon atoms, preferably 12 to 18 carbon atoms. Examples of soaps are sodium stearate, sodium palmitate, sodium laurate, sodium myristate, sodium behenate, potassium stearate, potassium palmitate, sodium myristate, sodium oleate, salts of hydroxy fatty acids, for example salts of 12-hydroxystearic acid or salts of 16-hydroxyhexadecanoic acid.

Preferred components B are lauric acid, palmitic acid, stearic acid, and mixtures and salts thereof. The fatty acids are generally used in order to impart a refatting and caring skin feel to the composition.

A composition according to the invention can preferably have a fraction of component B of 25.0-50.0% by weight, based on the composition, and particularly preferably of 25.0-35.0% by weight, based on the composition.

A composition according to the invention comprises at least one acyl isethionate as component C.

Within the context of a preferred embodiment, the composition according to the invention comprises at least one acyl isethionate of formula (II) as component C:

R—CO—O—CHR¹—CHR²—SO₃X (II)

in which

R is the alkyl radical of a C₈-C₁₈-fatty acid,

R¹ and R², independently of one another, are H or CH₃, preferably H, and X is a cation, preferably an alkali metal cation, in particular Na.

These include acyl isethionates and methyl acyl isethionates with a C₈-C₁₈ acyl radical and mixtures thereof, preferably sodium salts thereof. Particular preference is given to sodium lauroyl isethionate or sodium cocoyl isethionate.

As a result of adding component C, a composition according to the invention exhibits a particularly good stability.

A composition according to the invention can preferably have a fraction of component C of 28.0-50.0% by weight, based on the composition and particularly preferably of 40.0-48.0% by weight, based on the composition.

Furthermore, a composition according to the invention comprises sodium isethionate, the sodium salt of 2-hydroxyethanesulfonic acid, as component D.

A composition according to the invention can preferably have a fraction of component D of 2.0-10.0% by weight, based on the composition, and particularly preferably of 3.0-6.0% by weight, based on the composition.

As a result of adding sodium isethionate to the composition according to the invention, the grittiness, for example, of the composition can be adjusted.

An important criterion when assessing soap bars is the question whether the soap feels soft, or rough, on the skin during use. The roughness depends on the grittiness of the soap and can be adjusted through the use of sodium isethionate. The addition of sodium isethionate, particularly in the amount described here, can lead in a composition according to the invention to an improvement in grittiness and thus to a reduction in roughness.

A composition according to the invention further comprises water as component E.

Preferably, a composition according to the invention has a water content of 1.0-10.0% by weight, particularly preferably of 2.0-7.0% by weight, based on the composition.

In one embodiment, the composition according to the invention comprises one or more additives F, preferably from the group consisting of preservatives, fragrances, dyes, further surfactants, cationic polymers, pigments, superfatting agents, antimicrobial and biogenic active ingredients, moisturizing agents, stabilizers, acids, alkalis, and mixtures thereof, preferably in amounts of 1.0-20.0% by weight, particularly preferably of 2.0-15.0% by weight and in particular of 3.0-10.0% by weight, in each case based on the total composition.

Suitable preservatives are all of the preservatives listed in the relevant annex of the European cosmetics legislation, for example phenoxyethanol, benzyl alcohol, parabens, benzoic acid and sorbic acid, with particularly well suited examples being 1,3-bis(hydroxymethyl)-5,5-dimethylimidazolidine-2,4-diones (Nipaguard® DMDMH), piroctone olamine, methylisothiazolinone or mixtures thereof, preferably piroctone olamine and/or methyl isothiazolinone.

Fragrances and perfume substances or oils that can be used are individual odorant compounds, e.g. the synthetic products of the ester, ether, aldehyde, ketone, alcohol and hydrocarbon type. Odorant compounds of the ester type are e.g. benzyl acetate, phenoxyethyl isobutyrate, p-tert-butyl cyclohexylacetate, linalyl acetate, dimethyl benzyl carbonyl acetate, phenyl ethyl acetate, linalyl benzoate, benzyl formate, ethyl methylphenylglycinate, allyl cyclohexyl propionate, styrallyl propionate and benzyl salicylate. The ethers include, for example, benzyl ethyl ether, the aldehydes include e.g. the linear alkanals having 8 to 18 carbon atoms, citral, citronellal, citronellyloxyacetaldehyde, cyclamen aldehyde, hydroxycitronellal, lillial and bourgeonal, the ketones include e.g. the ionones, alpha-isomethylionone and methyl cedryl ketone, the alcohols include anethole, citronellol, eugenol, geraniol, linalol, phenylethyl alcohol and terpineol, the hydrocarbons include primarily the terpenes and balsams. Preference is given to using mixtures of different fragrances which together produce a pleasant scent note.

Perfume oils can also contain natural odorant mixtures, as are accessible from vegetable or animal sources, e.g. pine, citrus, jasmine, lily, rose or ylang-ylang oil. Essential oils of relatively low volatility, which are mostly used as aroma components, are also suitable as perfume oils, e.g. sage oil, chamomile oil, clove oil, Melissa oil, mint oil, cinnamon leaf oil, linden blossom oil, juniper berry oil, vetiver oil, olibanum oil, galbanum oil and ladanum oil.

Suitable dyes are in principle all dyes which are approved for cosmetics use; these are listed in the corresponding annexes of the European cosmetics legislation.

For example, the dyes and pigments present may either be organic or inorganic dyes. Also used advantageously are pearlescent pigments, e.g. pearl essence (guanine/hypoxanthine mixed crystals from fish scales) and mother-of-pearl (ground mussel shells), monocrystalline pearlescent pigments such as e.g. bismuth oxychloride (BiOCl), layer-substrate pigments, e.g. mica/metal oxide, silver-white pearlescent pigments of TiO₂, interference pigments (TiO₂, varying layer thickness), color luster pigments (Fe₂O₃) and combination pigments (TiO₂/Fe₂O₃, TiO₂/Cr₂O₃, TiO₂/Prussian blue TiO₂/carmine).

The amount of dyes and pigments in the compositions according to the invention is generally 0.1-2.0% by weight, based on the total weight of the finished compositions.

Further surfactants can in principle be all anionic, cationic or amphoteric surfactants that are cosmetically suitable.

Suitable anionic surfactants can be selected from the group of alkyl sulfates and alkyl ether sulfates.

Preferred alkyl sulfates are the C₈-C₂₀-alkyl sulfates, in particular the linear C₈-C₂₀-alkyl sulfates, preferably in the form of their sodium, potassium or ammonium salts. Examples of alkyl sulfates are lauryl sulfate, cocoalkyl sulfate and tallow alkyl sulfate. Particular preference is given to lauryl sulfate.

Preferred alkyl ether sulfates are the C₈-C₂₀-alkyl ether sulfates, particularly preferably the linear C₈-C₂₀-alkyl ether sulfates, in particular the alkyl glycol ether sulfates derived from the ethoxylated fatty alcohols, in the form of their sodium, potassium or ammonium salts. Examples of alkyl ether sulfates are lauryl ether sulfate, cocoalkyl ether sulfate and tallow alkyl ether sulfate. Particular preference is given to lauryl ether sulfate. Examples of glycol ether sulfates are lauryl triethylene glycol ether sulfate, cocoalkyltriethylene glycol ether sulfate and tallow alkylhexaethylene glycol ether sulfate. Particular preference is given to lauryl glycol ether sulfate, for example lauryl diethylene glycol ether sulfate or lauryl triethylene glycol ether sulfate, specifically in the form of the sodium salts.

A particularly preferred anionic surfactant is sodium lauryl ether sulfate.

In a further embodiment of the invention, the composition can comprise one or more N-acylamino acid surfactants as anionic surfactants. Within the scope of a preferred embodiment, the amino acid radical of such N-acylamino acid surfactants is selected from the group consisting of proteinogenic amino acids, N-alkylated derivatives thereof, and mixtures thereof.

Particularly preferred N-acylamino acid surfactants are acyl glycinates, acyl alaninates, acyl aspartates, acyl glutamates, acyl sarcosinates or mixtures thereof. The N-acylamino acid surfactants are very particularly preferably selected from the group consisting of acyl glycinate, acyl aspartate, acyl glutamate, acyl sarcosinate and mixtures thereof.

The N-acylamino acid surfactants very particularly preferably consist of at least one C₈-C₂₂-acylated amino acid, in particular N-alkylated derivatives thereof. Preference is given to the corresponding lauroyl or cocoyl derivatives of the amino acids.

Particular preference is therefore given to sodium cocoyl glycinate, potassium cocoyl glycinate, sodium lauroyl glycinate, potassium lauroyl glycinate, sodium cocoyl glutamate, sodium lauroyl glutamate, sodium cocoyl aspartate, sodium lauroyl aspartate and sodium lauroyl sarcosinate.

Suitable cationic surfactants are substituted or unsubstituted straight-chain or branched quaternary ammonium salts of the type R¹N(CH₃)₃X, R¹R²N(CH₃)₂X, R¹R²R³N(CH₃)X or R¹R²R³R⁴NX. The radicals R¹, R², R³ and R⁴ can preferably be, independently of one another, unsubstituted alkyl with a chain length between 8 and 24 carbon atoms, in particular between 10 and 18 carbon atoms, hydroxyalkyl with 1 to 4 carbon atoms, phenyl, C₂- to C₁₈-alkenyl, C₇- to C₂₄-aralkyl, (C₂H₄O)ₓH, where x is from 1 to 3, alkyl radicals containing one or more ester groups, or cyclic quaternary ammonium salts. X is a suitable anion. Preference is given to (C₈-C₂₂)-alkyltrimethylammonium chloride or bromide, particularly preferably cetyltrimethylammonium chloride or bromide, di(C₈-C₂₂)-alkyldimethylammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, particularly preferably distearyldimethylammonium chloride, di(C₈-C₂₂)-alkylamidopropyltrimethylammonium chloride and methosulfate.

Suitable nonionic surfactants are, for example, the following compounds:

Polyethylene, polypropylene and polybutylene oxide condensates of alkylphenols. These compounds comprise the condensation products of alkylphenols with a C₆- to C₂₀-alkyl group, which can either be linear or branched, and also with alkene oxides. These surfactants are referred to as alkylphenol alkoxylates, e.g. alkylphenol ethoxylates.

Condensation products of aliphatic alcohols with 1 to 25 mol of ethylene oxide. The alkyl or alkenyl chain of the aliphatic alcohols can be linear or branched, primary or secondary, and generally comprises 8 to 22 carbon atoms. Particular preference is given to the condensation products of C₁₀- to C₂₀-alcohols with 2 to 18 mol of ethylene oxide per mole of alcohol. The alcohol ethoxylates can have a narrow (“Narrow Range Ethoxylates”) or a broad homolog distribution of the ethylene oxide (“Broad Range Ethoxylates”). Examples of commercially available nonionic surfactants of this type are Tergitol® 15-S-9 (condensation product of a linear secondary C₁₁-C₁₅-alcohol with 9 mol of ethylene oxide), Tergitol® 24-L-NMW (condensation product of a linear primary C₁₂-C₁₄-alcohol with 6 mol of ethylene oxide with a narrow molecular weight distribution). This product class likewise includes the Genapol® grades from Clariant.

Condensation products of ethylene oxide with a hydrophobic base, formed by condensation of propylene oxide with propylene glycol. The hydrophobic moiety of these compounds preferably has a molecular weight between 1500 and 1800. The addition of ethylene oxide onto this hydrophobic moiety leads to an improvement in water solubility. The product is liquid up to a polyoxyethylene content of approx. 50% of the total weight of the condensation product, which corresponds to a condensation with up to approx. 40 mol of ethylene oxide. Commercially available examples of this product class are the Pluronic® grades from BASF and the Genapol® PF grades from Clariant.

Condensation products of ethylene oxide with a reaction product of propylene oxide and ethylenediamine. The hydrophobic unit of these compounds consists of the reaction product of ethylenediamine with excess propylene oxide and generally has a molecular weight of 2500 to 3000. Ethylene oxide is added onto this hydrophobic unit to a content of 40 to 80% by weight of polyoxyethylene and a molecular weight of 5000 to 11 000. Commercially available examples of this compound class are the Tetronic® grades from BASF and the Genapol® PN grades from Clariant.

The nonionic surfactants are preferably fatty alcohol ethoxylates (alkyl polyethylene glycols); alkylphenol polyethylene glycols; fatty amine ethoxylates (alkylaminopolyethylene glycols); fatty acid ethoxylates (acyl polyethylene glycols); polypropylene glycol ethoxylates (Pluronics®); fatty acid alkanolamides, (fatty acid amide polyethylene glycols); sucrose esters; sorbitol esters and sorbitan esters and polyglycol ethers thereof, and C₈-C₂₂-alkyl polyglucosides.

Furthermore, the compositions according to the invention can comprise amphoteric surfactants. These can be described as derivatives of long-chain secondary or tertiary amines which have an alkyl group having 8 to 18 carbon atoms and in which a further group is substituted with an anionic group which imparts the water solubility, thus e.g. with a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates as alkali metal and mono-, di- and trialkylammonium salts. Suitable further surfactants are also amine oxides. These are oxides of tertiary amines with a long-chain group of 8 to 18 carbon atoms and two mostly short-chain alkyl groups having 1 to 4 carbon atoms. Preference is given here for example to the C₁₀- to C₁₈-alkyl dimethylamine oxides and fatty acid amidoalkyl dimethylamine oxides.

In a preferred embodiment, the compositions according to the invention additionally also comprise, as foam-boosting agents, cosurfactants from the group of alkylamidobetaines, aminopropionates, aminoglycinates, imidazoliniumbetaines and sulfobetaines, amine oxides, fatty acid alkanolamides and polyhydroxyamides.

Preferred further surfactants are: ethoxylated and propoxylated fatty alcohols, ethoxylated and propoxylated triglycerides such as PEG-40 hydrogenated castor oil or fatty acid esters, ether carboxylates, alkyl polyglucosides, olefinsulfonates, sec-alkylsulfonates and taurates.

Suitable cationic polymers are those known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37 & mineral oil & PPG trideceth (Salcare SC95), PVP-dimethylaminoethyl methacrylate copolymer, guar hydroxypropyltriammonium chlorides, and also calcium alginate and ammonium alginate. It is also possible to use cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as e.g. amidomethicones; copolymers of adipic acid and dimethylaminohydroxypropyldiethylenetriamine; polyaminopolyamide and cationic chitin derivatives, such as, for example, chitosan.

Superfatting agents that can be used are preferably lanolin and lecithin, nonethoxylated and polyethoxylated or acylated lanolin and lecithin derivatives, polyol fatty acid esters, mono-, di- and triglycerides and/or fatty acid alkanolamides, and ethoxylated triglycerides such as PEG-7 glyceryl cocoate or mixtures of glyceryl oleate with alkyl polyglucosides.

Used on antimicrobial active ingredients are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxy diphenyl ether (triclosan), phenoxyethanol, 1,5-pentanediol, 1,6-hexanediol, 3,4,4′-trichlorocarbanilide (triclocarban), diaminoalkylamide, for example L-Iysine-hexadecylamide, citrate heavy metal salts, salicylates, piroctose, in particular zinc salts, pyrithiones and heavy metal salts thereof, in particular zinc pyrithione, zinc phenolsulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and octopirox, iodopropynyl butylcarbamate, methylchloroisothiazolinone, methylisothiazolinone, methyldibromo-glutaronitrile, AgCl, chloroxylenol, Na salt of diethylhexylsulfosuccinate, sodium benzoate, and phenoxyethanol, benzyl alcohol, phenoxyisopropanol, parabens, preferably butyl-, ethyl-, methyl- and propylparaben, and Na salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of sorbic acid and combinations of these active substances.

The compositions according to the invention can furthermore comprise biogenic active ingredients selected from plant extracts, such as, for example, aloe vera, and also local anesthetics, antibiotics, antiphlogistics, antiallergics, corticosteroids, sebostatics, Bisabolol®, Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and vitamin E and/or derivatives thereof.

Moisturizing substances are for example isopropyl palmitate, glycerol, diglycerol and/or sorbitol. Particular preference is given to glycerol.

Preferably, a composition according to the invention has a pH of 3 to 9, particularly preferably of 5 to 8.

The acids or alkalis used for adjusting the pH are preferably mineral acids, in particular HCl, inorganic bases, in particular NaOH or KOH, or organic acids, in particular citric acid or lactic acid.

A composition according to the invention can also have complexing agents, via which for example alkali ions can be complexed and thus the stability of the composition can be improved. Typical complexing agents are for example EDTA (ethylenediaminetetraacetate) and nitrilotriacetic acid. Preference is given to using EDTA as complexing agent.

Besides water, a composition according to the invention can also comprise at least one further solvent. Within the context of the present invention, a solvent is preferably understood as meaning protic solvent such as C₁-C₈-alcohols, in particular C₁-C₆-alcohols, ethylene glycol, diethylene glycol, triethylene glycol or mixtures thereof, with water and/or ethanol or water and/or methanol in particular being preferred. From the C₁-C₆-alcohols, methanol, ethanol, isopropanol, n-butanol or sec-butanol are preferred.

Furthermore, the compositions according to the invention can comprise film formers which, depending on the intended use, are selected from salts of phenylbenzimidazole sulfonic acid, water-soluble polyurethanes, for example C₁₀-polycarbamyl polyglyceryl ester, polyvinyl alcohol, polyvinylpyrrolidone copolymers such as PVP/hexanedecene or PVP/eicosene copolymer, for example vinylpyrrolidone/vinyl acetate copolymer, water-soluble acrylic acid polymers/copolymers and esters or salts thereof, for example partial ester copolymers of acrylic/methacrylic acid and polyethylene glycol ethers of fatty alcohols, such as acrylate/-steareth-20 methacrylate copolymer, water-soluble cellulose, for example hydroxymethyl cellulose, hydroxyethyl cellulose, hydroxypropyl cellulose, water-soluble quaterniums, polyquaterniums, carboxyvinyl polymers, such as carbomers and salts thereof, polysaccharides, for example polydextrose and glucan, vinyl acetate/crotonate, available for example under the trade name Aristoflex® A 60 (Clariant), and polymeric amine oxides, for example representatives available under the trade names Diaformer Z-711, 712, 731, 751.

A composition according to the invention can also have at least one oil body. The oil bodies can advantageously be selected from the groups of natural and synthetic fatty bodies, preferably triglycerides, esters of fatty acids with alcohols of low carbon number, e.g. with isopropanol, propylene glycol or glycerol, or esters of fatty alcohols with alkanoic acids of low carbon number or with fatty acids or from the group of alkyl benzoates, and also natural or synthetic hydrocarbon oils and silicone oils.

Particular preference is given to triglyceride oils such as sunflower oil and soybean oil; particular preference is likewise given to petrolatum (Vaseline).

Of suitability are preferably triglycerides of linear or branched, saturated or unsaturated, optionally hydroxylated, C₈-C₃₀-fatty acids, in particular vegetable oils, such as sunflower oil, corn oil, soybean oil, rice oil, jojoba oil, babusscu oil, pumpkin oil, grape seed oil, sesame oil, walnut oil, apricot oil, orange oil, wheat germ oil, peach kernel oil, macadamia oil, avocado oil, sweet almond oil, lady's smock oil, castor oil, olive oil, peanut oil, rapeseed oil and coconut oil, as well as synthetic triglyceride oils, e.g. the commercial product Myritol® 318. Hydrogenated triglycerides are also preferred according to the invention. Oils of animal origin, for example beef tallow, perhydrosqualene, lanolin, can also be used.

Oil bodies further preferred according to the invention are the benzoic acid esters of linear or branched C₈₋₂₂-alkanols, e.g. the commercial products Finsolv® SB (isostearyl benzoate), Finsolv® TN (C₁₂-C₁₅-alkyl benzoate) and Finsolv® EB (ethylhexyl benzoate).

A further class of oil bodies preferred according to the invention are dialkyl ethers with in total 12 to 36 carbon atoms, in particular with 12 to 24 carbon atoms, such as e.g. di-n-octyl ether (Cetiol® OE), di-n-nonyl ether, di-n-decyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, di-3-ethyldecyl ether, tert-butyl-n-octyl ether, isopentyl n-octyl ether and 2-methylpentyl n-octyl ether, and also di-tert-butyl ether and diisopentyl ether.

Likewise of suitability are branched saturated or unsaturated fatty alcohols having 6-30 carbon atoms, e.g. isostearyl alcohol, and also guerbet alcohols.

A further class of preferred oil bodies are dicarboxylic acid esters of linear or branched C₂-C₁₀-alkanols, such as di-n-butyl adipate (Cetiol® B), di-(2-ethylhexyl) adipate and di-(2-ethylhexyl) succinate, and also diol esters such as ethylene glycol dioleate, ethylene glycol diisotridecanoate, propylene glycol di-(2-ethylhexanoate), propylene glycol diisostearate, propylene glycol dipelargonate, butanediol diisostearate and neopentyl glycol dicaprylate, and diisotridecyl acetate.

Likewise preferred oil bodies are symmetrical, asymmetrical or cyclic esters of carbonic acid with fatty alcohols, glycerol carbonate or dicaprylyl carbonate (Cetiol® CC).

A further class of preferred oil bodies are the esters of dimers of unsaturated C₁₂-C₂₂-fatty acids (dimer fatty acids) with monohydric linear, branched or cyclic C₂-C₁₈-alkanols or with polyhydrically linear or branched C₂-C₆-alkanols.

A further class of preferred oil bodies are hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and also hexadecane, isohexadecane, paraffin oils, isoparaffin oils, e.g. the commercial products of the Permethyl® series, squalane, squalene, and alicyclic hydrocarbons, e.g. the commercial product 1,3-di-(2-ethylhexyl)cyclohexane (Cetiol® S), ozocerite and ceresin.

Preferred compositions according to the invention comprise:
at least one N-alkyl-N-acylglucamine, where the N-alkyl-N-acylglucamine comprises at least one C₁₂- and/or C₁₄-acyl group,
at least one fatty acid and/or soap, where the fatty acid is preferably stearic acid,
sodium cocoyl isethionate or sodium lauroyl isethionate as component C and
sodium isethionate as component D,
where more than 70% by weight of the N-alkyl-N-acylglucamines contain at least one C₁₂- and/or C₁₄-acyl group.

The preferred embodiment can be used either as combibar or syndet bar.

Further preferred compositions according to the invention comprise:
at least one N-alkyl-N-acylglucamine, where the N-alkyl-N-acylglucamine is derived from coconut oil,
at least one fatty acid and/or soap, where the fatty acid is preferably stearic acid,
sodium cocoyl isethionate as component C and
sodium isethionate as component D,

where more than 70% by weight of the N-alkyl-N-acylglucamines contain at least one C₁₂- and/or C₁₄-acyl group.

The preferred compositions can be used either as combibar or syndet bar.

Within the context of a preferred embodiment, the composition according to the invention is a cosmetic or dermatological composition.

The invention further provides the use of the composition according to the invention as soap bar. Preferably, the soap bar is a combibar or syndet bar.

The invention further provides the use of the composition according to the invention for the treatment or care of the skin.

The invention further provides the use of the composition according to the invention for the treatment or care of the hair.

The invention further provides a process for producing a composition, where the components A, B, C, D, E and optionally F are brought into contact with one another. The preparation can be carried out in accordance with customary methods. Here, the components of the composition according to the invention are mixed together, which can take place e.g. by means of kneading. The resulting mass can be converted to the desired shape e.g. by extrusion, cutting, bar pressing or molding. Production takes place preferably at elevated temperatures, in particular at temperatures between 40° C. and 90° C.

The examples below serve to illustrate the invention, but without limiting it thereto.

### EXAMPLES

The N-alkyl-N-acylglucamines described below were prepared in accordance with EP 0 550 637 from the corresponding fatty acid methyl esters and N-acylglucamide in the presence of 1,2-propylene glycol as solvent, and obtained in the form of a solid consisting of active substance, i.e. N-alkyl-N-acylglucamine, and 1,2-propylene glycol (all data in % by weight).

TABLE 1
Preparation examples for N-alkyl-N-acylglucamine

| | | | Active | 1,2- | Melting |
|---|---|---|---|---|---|
| Preparation | | | substance | Propylene | point |
| example | Methylester | Triglyceride | (%) | glycol (%) | (° C.) |
| 1 | C12/14 | — | 90 | 10 | 85 |
| | (C12: 70%, | | | | |
| | C14: 30%) | | | | |
| 2 | C16/18 | — | 80 | 20 | 65 |
| | (C16: 60%; | | | | |
| | C18: 40%) | | | | |
| 3 | — | Coconut oil | 90 | 10 | 55 |
| | | (C8: 6% | | | |
| | | C10: 6% | | | |
| | | C12: 48% | | | |
| | | C14: 20% | | | |
| | | C18: 2% | | | |
| | | C18′: 8%) | | | |

C18′ means an oleoyl radical.
The melting point was determined by means of a Kofler hot bench.
Compositions (soap bars) (all percentages are % by weight based on the composition)

Composition No. 1 (Use as Syndet Bar)

46.2% Hostapon SCI 65C (comprises 65% sodium cocoyl isethionate and

35% stearic acid)

25.2% stearic acid

1% glucamine (as per Table 1)

10% polyglycol 20000P

5% Hostapon SI (57% solution of sodium isethionate in water)

0.05% etidronic acid

2.0% corn starch

0.3% titanium dioxide

9.25% water (deionized)

1% citric acid

Composition No. 2 (Standard for Syndet Soap)

47.2% Hostapon SCI 65C (comprises 65% sodium cocoyl isethionate and

35% stearic acid)

25.2% stearic acid

0% N-alkyl-N-acylglucamine (as per Table 1)

10% polyglycol 20000P

5% Hostapon SI (57% solution of sodium isethionate in water)

0.05% etidronic acid

2.0% corn starch

0.3% titanium dioxide

9.25% water (demineralized)

1% citric acid

Preparation of the Compositions

The compositions were prepared in a soap machine which consists of a laboratory kneader (model LTK 3R), roller flocker (W3K-32 R) and a screw extruder (SVZ 75R).

TABLE 2
Properties of the compositions compared to composition 2

| No. of | | Amount of | |
|---|---|---|---|
| the soap | Composition | lather | Hardness |
| 1 | Composition 1 | More lather | Brittle |
| | (N-alkyl-N-acylglucamine as | | |
| | per preparation example 2) | | |
| 2 | Composition 1 | More lather | Increased |
| | (N-alkyl-N-acylglucamine as | | hardness |
| | per preparation example 3) | | |
| 3 | Composition 1 | More lather | Increased |
| | (N-alkyl-N-acylglucamine as | | hardness |
| | per preparation example 1) | | |

The properties were ascertained by three test persons by means of handwashing with the compositions. The hardness was ascertained by manual evaluation.

The amount of lather and the hardness were determined compared to composition 2 which comprised no N-alkyl-N-acylglucamines.

Result

N-Alkyl-N-acylglucamines as per preparation examples 1 and 3 increase the amount of lather in composition 1 and lead to an increased hardness compared to the standard (composition 2).

Analysis of the Dissolution Behavior (“Mushiness”) of the Compositions as Soap Bars

Carrying Out the Analysis

Each soap bar is placed into a vessel of identical volume. The position of the soap bar is kept similar as far as possible. After positioning the soap bar, the vessel is filled with a specified amount of water, which is identical for each vessel and therefore for each soap bar. Each vessel is sealed with aluminum foil in order to avoid evaporation and stored at room temperature. After 1, 4, 5, 9 and 14 days, the length of each soap bar is measured. The results are shown in Table 3.

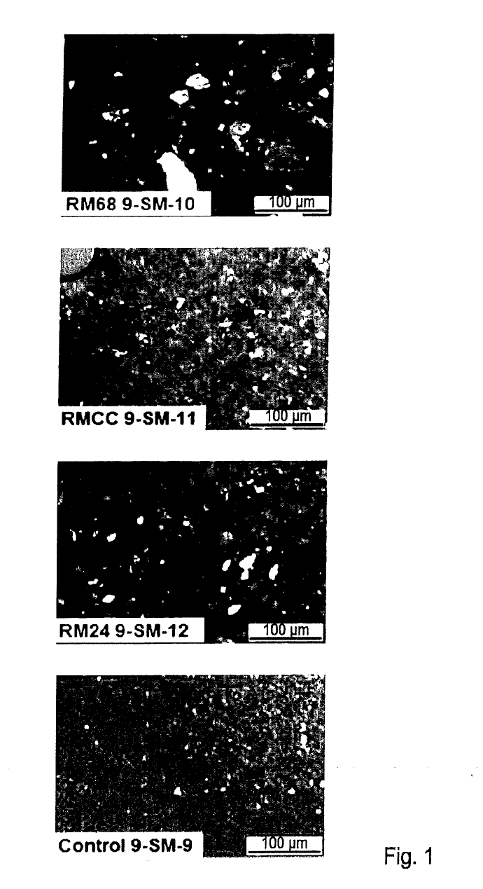

To determine the mechanism which leads to the dissolution (“mushiness”) of the soap bars, after one day the surface of the soap bar is analyzed by means of microscopy using polarized light. The results are shown in FIG. 1.

The soaps 1, 2 and 3 according to the invention with a lamellar structure of the surface have a slower dissolution in water here, which in practice leads to a longer durability of the syndet soap for the consumer. By contrast, the control sample (composition 2, control 9-SM-9) already shrunk considerably after one day.

TABLE 3
Data for the length of the soap bar in [cm]
as a function of the composition and number of days.
The soaps 1, 2 and 3 correspond to those from Table 2.

| | 0 | 1 | 4 | 5 | 9 | 14 |
|---|---|---|---|---|---|---|
| Soap No. | days | day | days | days | days | days |
| 1 (RM68 9-SM-10) | 6.0 | 5.5 | 3.5 | 0 | | |
| 2 (RMCC 9-SM-11) | 5.5 | 5.0 | 0 | | | |
| 3 (RM24 9-SM-12) | 6.0 | 5.5 | 5.0 | 4.2 | 4.0 | 0 |
| Composition 2 | 6.0 | 4.5 | 0 | | | |
| (control 9-SM-9) | | | | | | |

In soaps of composition 1, in particular N-alkyl-N-acylglucamines as per preparation example 1 with a C_{12/14} chain cut significantly slow the dissolution of the syndet soap.

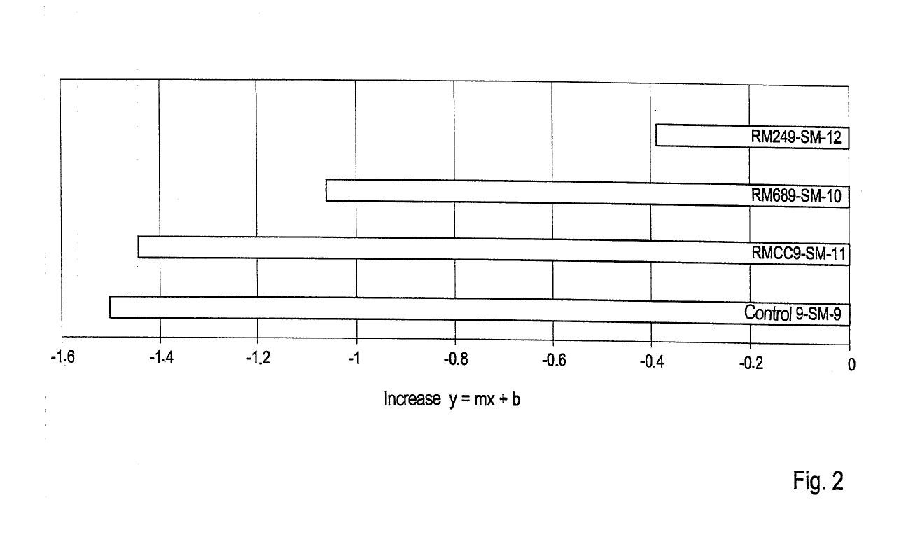

The increases in the results from Table 3 are shown in FIG. 2.

1. A composition comprising:
at least one N-alkyl-N-acylglucamine as component A,
at least one fatty acid and/or soap as component B,
at least one acyl isethionate as component C,
sodium isethionate as component D,
water as component E,
optionally at least one further additive as component F,
where more than 20% by weight, of the N-alkyl-N-acylglucamines contain at least one C₁₂ and/or C₁₄ and/or C₁₆ and/or C₁₈-acyl group.

2. The composition as claimed in claim 1, comprising:
0.5-5.0% by weight, of the composition of component A,
25.0-50.0% by weight, of the composition of component B,
28.0-50.0% by weight, of the composition of component C,
2.0-10.0% by weight, of the composition of component D.

3. The composition as claimed in claim 1, where more than 90% by weight of the N-alkyl-N-acylglucamines contain at least one C₁₂ and/or C₁₄ and/or C₁₆ and/or C₁₈-acyl group.

4. The composition as claimed in claim 1, where the at least one component A consists of one or more C₈-C₂₂-N-alkyl-N-acylglucamines.

5. The composition as claimed in claim 1, where the component A consists of one or more C₈-C₂₂-N-methyl-N-acylglucamines.

6. The composition as claimed in claim 1, where the at least one component B comprises a C₁₂-C₁₈ fatty acid, salt thereof or a mixture thereof.

7. The composition as claimed in claim 1, where the at least one component B consists of lauric acid, palmitic acid, stearic acid, salts thereof or mixtures thereof.

8. The composition as claimed in claim 1, where the at least one component C consists of sodium lauroyl isethionate, sodium cocoyl isethionate or mixture thereof.

9. The composition as claimed in claim 1, where the at least one component F is selected from the group consisting of preservatives, fragrances, dyes, surfactants, cationic polymers, pigments, superfatting agents, antimicrobial and biogenic active ingredients, moisturizing agents, stabilizers, acids and alkalis.

10. A cosmetic or dermatological composition comprising the composition as claimed in claim 1.

11. A soap bar comprising the composition as claimed in claim 1.

12. A for treating or caring for the skin, the hair or skin and hair comprising the composition as claimed in claim 1.

13. A process for producing a composition comprising:
at least one N-alkyl-N-acylglucamine as component A,
at least one fatty acid and/or soap as component B,
at least one acyl isethionate as component C,
sodium isethionate as component D,
water as component E,
optionally at least one further additive as component F,
where more than 20% by weight, of the N-alkyl-N-acylglucamines contain at least one C₁₂ and/or C₁₄ and/or C₁₅ and/or C₁₈-acyl group where the components A, B, C, D, E and optionally F are brought into contact with one another.

14. The composition as claimed in claim 1 where more than 70% by weight, of the N-alkyl-N-acylglucamines contain at least one C₁₂ and/or C₁₄ and/or C₁₆ and/or C₁₈-acyl group.

15. The composition as claimed in claim 1, comprising:
1.0-3.0% by weight, of the composition of component A,
25.0-35.0% by weight, of the composition of component B,
40.0-48.0% by weight of the composition of component C,
3.0-6.0% by weight of the composition of component D.
